(19) United States
(12) Patent Application Publication
(10) Pub. No.: US 2008/0082170 A1
(43) Pub. Date: Apr. 3, 2008

(54) Apparatus and methods for surgical repair

(76) Inventor: Marc M. Peterman, Austin, TX (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/541,356

(22) Filed: Sep. 29, 2006

(51) Int. Cl. A61F 2/44; A61B 17/56

(52) U.S. Cl. 623/17.16; 606/60

(57) ABSTRACT

A mesh implant repairs annular defects in a spine. The mesh might include natural or synthetic or biocompatible materials. The mesh implant may be placed using peri-annular or intra-annular techniques. In one embodiment, the mesh has tails configured to thread through perforations made in the endplates of vertebral bodies above and below the affected annulus. The surgeon might place the implant using manual or instrument-assisted placement techniques.

### TECHNICAL FIELD

The inventive concepts relate generally to implants. More particularly, the invention concerns apparatus and methods relating to a mesh implant, such as a mesh implant for repairing disc defects, such as annular defects, in a spine, and associated methods and apparatus for implanting the mesh implant.

### BACKGROUND

Modern spine surgery often involves the use of spinal implants to correct or treat various spine disorders or to support the spine. Spinal implants may help, for example, to stabilize the spine, correct deformities of the spine, facilitate fusion, treat spinal fractures, or repair annular defects, for example, in herniated discs.

Specifically, with respect to repairing annular defects, several conventional approaches exist. Typical conventional approaches, however, use devices with rigid fasteners and materials. The rigid materials might have undesirable effects, such as contact with sensitive nearby tissues or injury to nerves.

Furthermore, existing intra-annular or sub-annular approaches entail deep placement, i.e., deep placement within or beneath the fibers of the annulus. A degenerated annulus, however, might not have the ability to retain the device. Placement of the device also entails a certain degree of nuclear tissue removal. Moreover, placement of the device typically increases the exposure of the body to foreign nucleus matter, typically resulting in an aggravated inflammation response. A need exists for an implant that allows for repairing annular defects without having the disadvantages of conventional approaches.

### SUMMARY

The disclosed novel concepts relate to apparatus and methods for repairing a disc defect, e.g., an annular defect, in a spine. In one exemplary embodiment, an implant includes a mesh that has a pair of tails configured to be threaded through a respective pair of perforations in two vertebral bodies of the spine.

In another exemplary embodiment, a method of repairing an annular defect in a spine uses a mesh implant. The mesh implant includes a mesh coupled to a pair of tails. The method includes threading one tail through a perforation in a vertebral body of the spine. The method further includes threading the second tail through another perforation in another vertebral body of the spine.

A third exemplary embodiment relates to an apparatus for placing a mesh implant in a spine. The mesh implant is configured to repair an annular defect in the spine. The apparatus includes a body, and a shaft coupled to the body. The apparatus further includes a plate configured to slide within the body, and at least one member configured to thread at least one tail of the mesh implant.

In yet another exemplary embodiment, an apparatus includes a mesh implant for repairing an annular defect in a spine. The mesh includes at least one therapeutic agent, or a combination of therapeutic agents, as desired. The therapeutic agent may include (but is not limited to) an anti-inflammatory agent, an anti-adhesive agent, and/or a pro-adhesive agent.

### BRIEF DESCRIPTION OF THE DRAWINGS

The appended drawings illustrate only exemplary embodiments of the invention and therefore should not be considered or construed as limiting its scope. Persons of ordinary skill in the art who have the benefit of the description of the invention appreciate that the disclosed inventive concepts lend themselves to other equally effective embodiments. Unless noted otherwise, in the drawings, the same numeral designators used in more than one drawing denote the same, similar, or equivalent functionality, components, or blocks.

FIG. 1 shows a herniated disc, suitable for repair by the disclosed mesh or patch implants.

FIG. 2 illustrates another view of a herniated disc, suitable for repair by the disclosed implants.

FIG. 3 depicts a mesh implant according to an exemplary embodiment of the invention used to repair a disc defect.

FIG. 4 shows the details of making a pair of perforations in vertebral body endplates according to an exemplary embodiment of the invention.

FIG. 5 illustrates peri-annular placement of a mesh implant to repair an annular defect according to an exemplary embodiment of the invention.

FIG. 6 depicts intra-annular placement of a mesh implant to repair an annular defect according to an exemplary embodiment of the invention.

FIG. 7 shows details of a mesh implant according to an illustrative embodiment of the invention.

FIG. 8 illustrates one technique for tying a knot in a mesh implant according to an exemplary embodiment of the invention.

FIG. 9 depicts one part of a manual technique for threading the tails of the mesh implant into the respective perforations or openings in the spine's vertebral bodies according to one illustrative embodiment of the invention.

FIG. 10 shows another part of a manual technique for threading the tails of the mesh implant into the respective perforations or openings in the spine's vertebral bodies according to one illustrative embodiment of the invention.

FIG. 11 illustrates an instrument for threading the tails of the mesh implant into the respective perforations or openings in the spine's vertebral bodies according to one illustrative embodiment of the invention.

FIG. 12 depicts details of the operation of the instrument shown in FIG. 11.

### DETAILED DESCRIPTION

The disclosed novel concepts relate to apparatus and methods for repairing a disc defect, e.g., an annular defect, in a spine, including a mesh implant and associated methods and apparatus for implanting the mesh implant. An implant system according to the disclosed concepts includes a mesh or patch implant, together with a tool or instrument for positioning or implanting the mesh implant within a patient's spine.

The inventive implants are a safe and elegant way of bolstering the posterior annulus and preventing recurring herniation. The implant includes an annular patch, which the surgeon applies to the nucleus pulposus, or nucleus, of a disc in a spine. Unlike conventional devices, the surgeon secures the implant to itself (ties the tails of the implant to the mesh, as described below in detail), rather than using rigid fasteners, such as screws, plugs, etc.

As persons of ordinary skill in the art understand, herniated discs result in release of nucleus matter. The inventive device retains the herniated nucleus pulposus, and may also allow the reintroduction of extruded nucleus pulposus materials into the disc space, rather than excising it. In addition, the implant might provide retention of other devices, such as nucleus replacement implants.

Furthermore, the device provides a mechanism for delivery of a therapeutic agent. The therapeutic agent might constitute medication, carried on and eluded by the device, or texture features to elicit a specific biologic response.

Surgeons may implant the device in a number of ways. For example, as described below in detail, surgeons may choose peri-annular placement or sub-annular placement. None of the placement techniques, however, relies exclusively on the annular fibers to retain the device. Rather, the surgeon uses positive anchoring in the tissues, as allowed by the patient's anatomy. The anchoring will often include anchoring directly to the bone of the vertebral endplates.

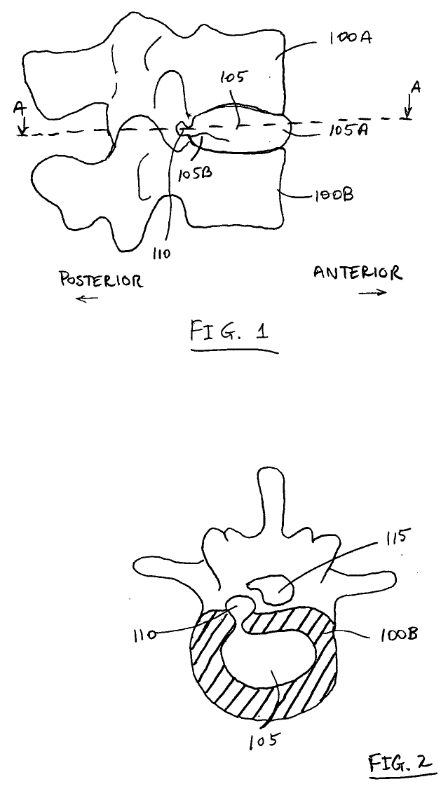

FIG. 1 shows a herniated disc, suitable for repair by the disclosed implants. Nucleus 105 resides between vertebral body 100A and vertebral body 100B. Nucleus 105 includes anterior annulus 105A and posterior annulus 105B. A posterior annular tear might result in release of the nucleus pulposus, thus producing a bulge 110 and possibly release of the nucleus pulposus, otherwise known as a herniated disc.

FIG. 2 illustrates another view of a herniated disc, suitable for repair by the disclosed implants. More specifically, FIG. 2 illustrates parts of the structures in FIG. 1, when sliced or viewed along line A-A, i.e., a top or transverse view.

When viewed from the top, one may observe that bulge 110 might come in contact with, or exert pressure to surrounding structures or tissues. For example, bulge 110 might compress neural element 115. As a result, the patient might experience pain, discomfort, or loss of function. In the case of a tear, the leakage of nucleus pulposus might result in a variety of problems and complications, as persons of ordinary skill in the art understand.

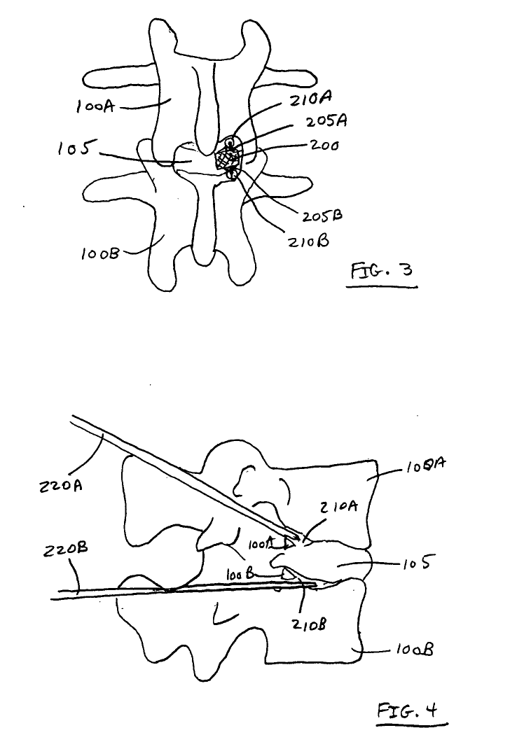

One may repair the disc defect (e.g., annular tear) by applying the inventive mesh implant. FIG. 3 depicts a mesh implant according to an exemplary embodiment of the invention used to repair a disc defect. The implant includes mesh 200, secured to vertebral body 100A and to vertebral body 100B.

Mesh 200 attaches to vertebral body 100A through perforation 210A, and to vertebral body 100B through perforation 210B. The respective positions of perforation 210A and perforation 210B depend on a number of factors, including the desired placement of mesh 200.

Generally speaking, one positions mesh 200 in a defective or damage area of the disc, e.g., over an annular tear. In one embodiment, perforation 210A and perforation 210B reside in the posterior margins of vertebral body 100A and vertebral body 100B, respectively. In other embodiments, one may select the precise positions of perforation 210A and perforation 210B depending on factors such as the desired position of mesh 200, the patient's anatomy, the nature of the defect in the disc, etc., as persons of ordinary skill in the art who have the benefit of the description of the invention understand.

To attach the mesh to the vertebral bodies, the surgeon uses a tunneling approach, as persons of ordinary skill in the art who have the benefit of the description of the invention understand. Tunneling in the posterior vertebral endplate anchors the tails of mesh 200 (as described below in detail), which in turn, anchors the mesh over the defect. As noted, the mesh provides reinforcement, which retains the extruded nucleus material.

More specifically, the surgeon makes perforation 210A in an endplate of vertebral body 100A. Similarly, the surgeon makes perforation 210B in an endplate of vertebral body 100B. FIG. 4 shows details of making perforation 210A and perforation 210B according to an exemplary embodiment of the invention.

The surgeon may use a variety of techniques and instruments to make perforation 210A and perforation 210B. Details of the instruments and of making perforations fall within the knowledge of persons of ordinary skill in the art who have the benefit of the description of the invention. For example, the surgeon may use a drill, a trochar, or a punch, as desired.

The exemplary technique shown in FIG. 4 uses a pair of trochars to make perforation 210A and perforation 210B. More specifically, the surgeon uses trochar 220A to make perforation 210A in an endplate of vertebral body 100A. The surgeon makes perforation 210A at a desired position, size, and angle (i.e., the angle of penetration of trochar 220A).

Similarly, the surgeon uses trochar 220B to make perforation 210B in an endplate of vertebral body 100B. The surgeon makes perforation 210B at a desired position, size, and angle. If desired, the surgeon may make perforation 210A and perforation 210B at complementary angles with respect to a horizontal (anterior-posterior or top or transverse) plane of annulus 105.

Generally, the size, angle, and location of perforation 210A and perforation 210B depend on a variety of factors, as persons of ordinary skill in the art who have the benefit of the description of the invention understand. The factors include the desired location of mesh 200 with respect to annulus 105, vertebral body 100A and vertebral body 100B, the patient's anatomy, the particular geometry and characteristics of mesh 200 and its tails (as described below), etc.

After performing the perforation procedure above, the surgeon attaches the implant. More specifically, the surgeon secures one end or region of mesh 200 to vertebral body 100A by using one or more knots 205A. Likewise, the surgeon uses one or more knots 205B to attach another end of mesh 200 to vertebral body 100B. As described below in detail, mesh 200 couples to a pair of tails. The surgeon uses a respective tail to tie knot 205A and knot 205B.

Note that knots constitute just one technique for securing the mesh implant in a desired location. One may use a variety of techniques to secure the mesh implant, as persons of ordinary skill in the art who have the benefit of the description of the invention understand, and as desired. As one example, one may use a crimping tool to crimp a sleeve or other suitable structure in order to secure the implant. As other examples, one may use fraction fits, braids, or cam locks, as desired.

Once attached, the device provides the benefits and functions described above. In other words, the implant provides retention of the nucleus pulposus, helps to avoid extrusion of the nucleus pulposus, and/or provides therapeutic agents, as described above. The implant also serves as a scaffold for scar tissue growth, further securing the implant in place.

As noted above, the surgeon may place or implant mesh 200 in a variety of positions with respect to annulus 105. For example, the surgeon may use a peri-annular placement or an intra-annular placement for mesh 200 and the implant generally.

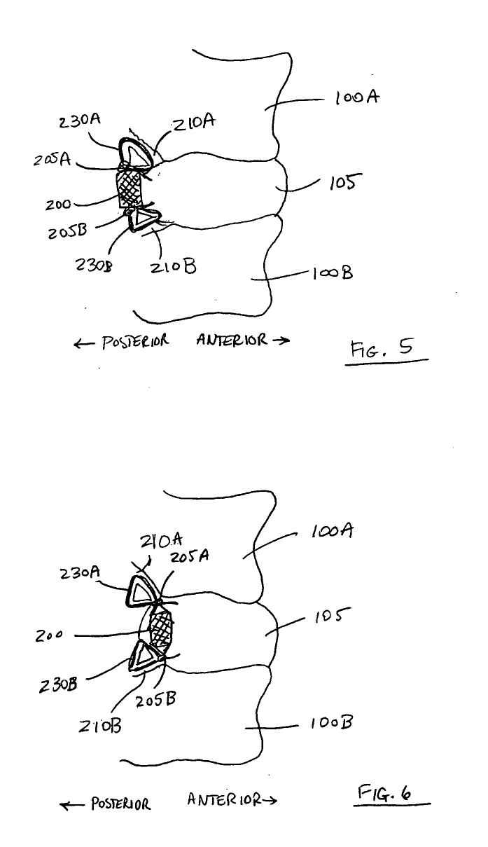

FIG. 5 illustrates peri-annular placement of a mesh implant according to an exemplary embodiment of the invention. Peri-annular placement refers to placement of mesh 200 and knots 205A and 205B on or above the surface of annulus 105. Put another way, with peri-annular placement, the surgeon implants mesh 200 and knots 205A and 205B superficially with respect to annulus 105.

In some cases of contained herniated nucleus pulposus, peri-annular placement of the mesh construct reinforces the posterior annulus without accessing the inter-discal space. This method of placement protects the surrounding materials from the harmful substances contained in the nucleus matter.

Furthermore, the inventive approach avoids worsening the annular defect, because the surgeon places the mesh on top of the defect. In fact, under some circumstances, the surgeon might even be able to push back the extruded nucleus matter into the defect. In cases where a disc bulge exists, the patch will reinforce the defective area without exposing the body to the nucleus pulposus.

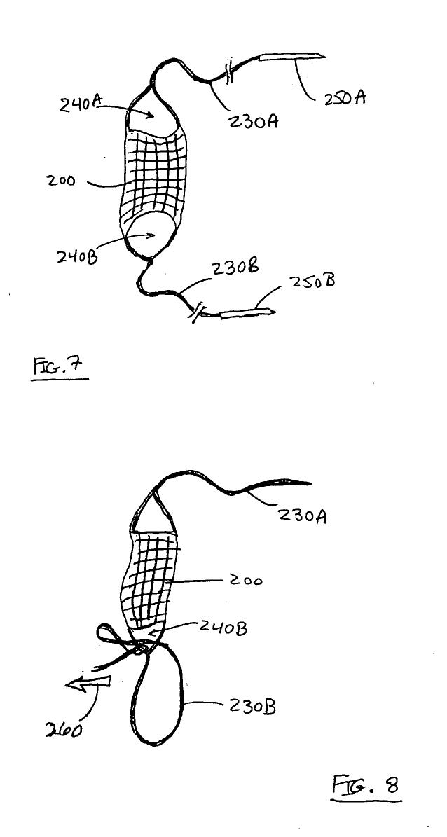

As noted above, mesh 200 couples to a pair of tails, shown as tail 230A and tail 230B in FIG. 5. Tail 230A couples or attaches to one end of mesh 200. Tail 230A couples or attaches to another end of mesh 200. FIG. 7 and its corresponding discussion provide details of the topology and construction of the mesh implant.

Tail 230A and tail 230B allow the surgeon to secure mesh 200 in a desired location. The surgeon may use tail 230A and tail 230B to tie the implant onto itself. In this manner, the surgeon can avoid using rigid fasteners, which have the disadvantages noted above.

FIG. 6 depicts intra-annular placement of a mesh implant according to an exemplary embodiment of the invention. Intra-annular (or sub-annular or deep) placement of the mesh implant results in a deeper placement of the implant with respect to annulus 105.

In peri-annular placement, tail 230A and tail 230B enter perforation 210A and 210B, respectively, from the posterior direction of respective vertebral body 100A and vertebral body 100B. In contrast, in intra-annular placement, the surgeon threads tail 230A and 230B so that they enter, respectively, perforation 210A and 210B from near annulus 105 and exit the posterior aspect of vertebral body 100A and vertebral body 100B, respectively.

More specifically, after making perforation 210A, the surgeon threads tail 230A through perforation 210A, starting with the end of perforation 210A nearer annulus 105. Thus, the free (i.e., the end not coupled to mesh 200 before placement of the implant) end of tail 230A enters perforation 210A near annulus 105, and exits perforation 210A at the posterior aspect of vertebral body 100A.

After threading through perforation 210A, the surgeon uses the free end of tail 230A to tie knot 205A. The surgeon might pull tail 230A to a desired degree of tension before or during the tying of knot 205A. Once the surgeon has finished tying knot 205A, the surgeon may cut off any excess portion of tail 230A.

Similarly, after making perforation 210B, the surgeon threads tail 230B through perforation 210B. The surgeon begins the threading from an end of perforation 210A that is closer to annulus 105. Thus, the free (i.e., the end not coupled to mesh 200 before placement of the implant) end of tail 230B enters perforation 210B near annulus 105. After threading, the end of tail 230B exits perforation 210A at the posterior of vertebral body 100B.

After threading through perforation 210B, the surgeon uses the free end of tail 230B to tie knot 205B. As noted above, the surgeon might pull tail 230B to a desired degree of tension before or during the tying of knot 205B. The surgeon may cut off any excess portion of tail 230B after finishing the tying of knot 205B.

FIG. 7 depicts details of a mesh implant according to an illustrative embodiment of the invention. The mesh implant includes mesh 200, tail 230A, and tail 230B. Optionally, the implant may include loop 240A and loop 240B. In addition, the implant may optionally include needle or guide 250A and needle or guide 250B.

Mesh 200 couples to tail 230A and tail 230B. The coupling may occur via loop 240A and loop 240B, respectively, or without them. Optional integral loop 240A and loop 240B facilitate the tying of knot 205A and 205B (see FIGS. 5 and 6), respectively (see FIG. 7 and its respective discussion).

One may fabricate mesh 200, tail 230A and tail 230B, and optional loop 240A and optional loop 240B from a variety of materials, as desired, and as persons of ordinary skill in the art who have the benefit of the description of the invention understand. The choice of material depends on the desired characteristics of those components, and the particular desired properties of the resulting implant.

Generally speaking, one may fabricate mesh 200 (and tails 230A and 230B and loops 240A and 240B, as desired) from a natural or synthetic pliable material. The material should be biocompatible and relatively pliable, although one may use a relatively rigid or semi-rigid material, as desired. Furthermore, the materials should encourage fibrous tissue encapsulation.

As an example of one material, one may use polyester to take advantage of its property of encouraging fibrous tissue encapsulation. Various methods are known to persons of ordinary skill in the art for using polyester to encourage tissue in growth. As a specific example, one may use Dacron. One may also coat (e.g., dry coat), impregnate, or micro-texture (or otherwise include or embed into), the material, for example, with therapeutic or medicated agents, to elicit the desired response.

Example of other materials or therapeutic or medicated agents that can be used include anti-inflammatory agents, anti-adhesive agents (to eliminate or reduce scar tissue), and/or pro-adhesive agents. Examples of anti-inflammatory agents are described in detail in U.S. patent application Ser. No. 11/455,401, titled “Improved Method of Treating Degenerative Spinal Disorders”, filed on Jun. 19, 2006, and incorporated herein by reference). Note, however, that in addition or instead one may use other suitable materials, as persons of ordinary skill in the art who have the benefit of the description of the invention understand. Furthermore, one may use a single material or agent or a combination of several materials or agents, as desired.

As noted, mesh 200 covers the herniated region or area of the disc or annulus 105. Mesh 200 might be permeable or impermeable, as desired. Generally speaking, mesh 200 need not be impermeable. Because mesh 200 buttresses and supports the herniated region, it prevents, or tends to prevent, the leakage and release of nucleus material. Furthermore, the patient's body will scar over during the healing process and thus helps to isolate and contain the nucleus material. Thus a two-stage process may occur in which a permeable mesh may act to seal the annulus: (1) the permeable mesh buttresses the insufficient tissue allowing the body to (2) create an impermeable fibrous scar.

As noted above, the mesh implant may optionally include needles or guides 250A and 250B coupled to an end of each respective tail (230A and 230B). Needle 250A and needle 250B facilitate the threading of respective tail 230A and tail 230B, the tying of knot 205A and knot 205B, respectively, or both.

Once the surgeon has performed the threading, the surgeon might cut off or detach or uncouple needle 250A before tying knot 205A (see FIGS. 5 and 6). Alternatively, the surgeon may use needle 250 in order to aid in tying knot 205A. After threading through perforation 210A, the surgeon may continue to use needle 250A to tie knot 205A. The surgeon may cut off or detach or uncouple needle 250A after tying knot 205A.

Similarly, once the surgeon has threaded tail 230B, the surgeon might cut off or detach or uncouple needle 250B before tying knot 205B (see FIGS. 5 and 6). Alternatively, to facilitate tying, after threading through perforation 210B, the surgeon may continue to use needle 250B to facilitate tying knot 205B. The surgeon may cut off or detach or uncouple needle 250B after tying knot 205B.

One may tie knots 205A and 205B in a variety of ways, as persons of ordinary skill in the art who have the benefit of the description of the invention understand. As one example, FIG. 8 depicts a technique for tying a knot in a mesh implant according to an exemplary embodiment of the invention.

To tie the knot, the surgeon threads the free end of tail 230B through loop 240B in the direction of arrow 260. After the first threading operation, the surgeon then may thread the end of tail 230B one or more times through loop 240B in order to produce a tighter or more secure knot. After the last threading, the surgeon may tie the free end of tail 230B using a conventional knot or surgical knot, as desired.

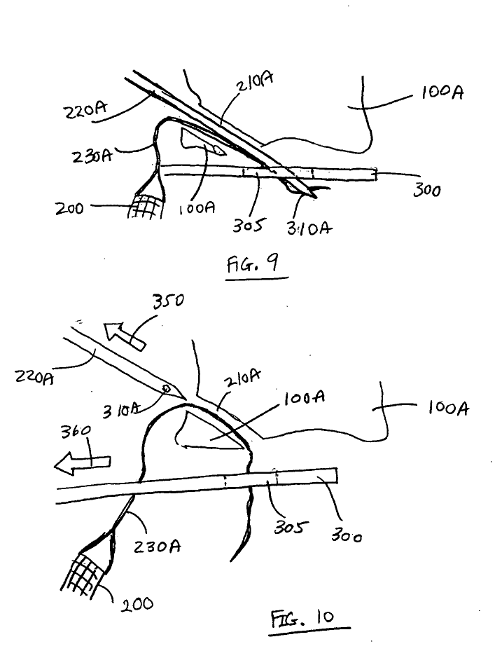

One may thread tails 230A and 230B through perforations or openings 210A and 210B, respectively, by using a manual approach, or by using an instrument-assisted approach. FIGS. 9 and 10 illustrate a manual technique of threading the tails 230A and 230B of the mesh implant.

In the technique illustrated, the surgeon uses trochar 220 and a plate or guide 300. Trochar 220 has an opening or hole 310A. Likewise, plate 300 has an opening or hole 305. Openings 310A and 305 facilitate the threading of tail 230A. Tail 230B of the mesh implant is similarly threaded. FIGS. 9 and 10 illustrate the threading of tail 230A through perforation 210A of vertebral body 100A. One may use a similar procedure to thread tail 230B through perforation 210B of vertebral body 100B, as persons of ordinary skill in the art who have the benefit of the description of the invention understand.

Referring to FIG. 9, the surgeon first threads tail 230A through opening 310A of trochar 220A. The surgeon then inserts trochar 220A into perforation 210A and into opening 305 of plate 300. As trochar 220A travels through perforation 210A of vertebral body 100A, it pulls or carries tail 230A through perforation 210A.

FIG. 10 illustrates how the surgeon completes the threading operation. Once trochar 220A and tail 230A are in their appropriate positions (though opening 305 of plate 300), the surgeon withdraws trochar 220A. The surgeon pulls trochar 220A in the direction generally shown by arrow 350, leaving the free end of tail 230A in opening 305 of plate 300.

Subsequently, the surgeon withdraws plate 300 from the patient's body, using a motion generally in the direction of arrow 360. As plate 300 moves in the direction shown by arrow 360, it pulls or withdraws the fee end of tail 230A from the patient's body. Once the surgeon has sufficiently withdrawn plate 300, he or she will have access to the free end of tail 230A. The surgeon may then use the retrieved free end of tail 230A to tie a knot and thus secure one end of mesh 200 in a desired location.

The surgeon may repeat the above technique for the other tail, i.e., tail 230B. Once the surgeon has retrieved tail 230B, he or she may tie another knot, thus securing the second end of mesh 200 in a desired location. At the conclusion of this procedure, mesh 200 will have a desired location with respect to the defect in annulus 105. As one alternative, the surgeon may thread both tail 230A and tail 230B through perforation 305 and retract both tails in direction 360 to secure them.

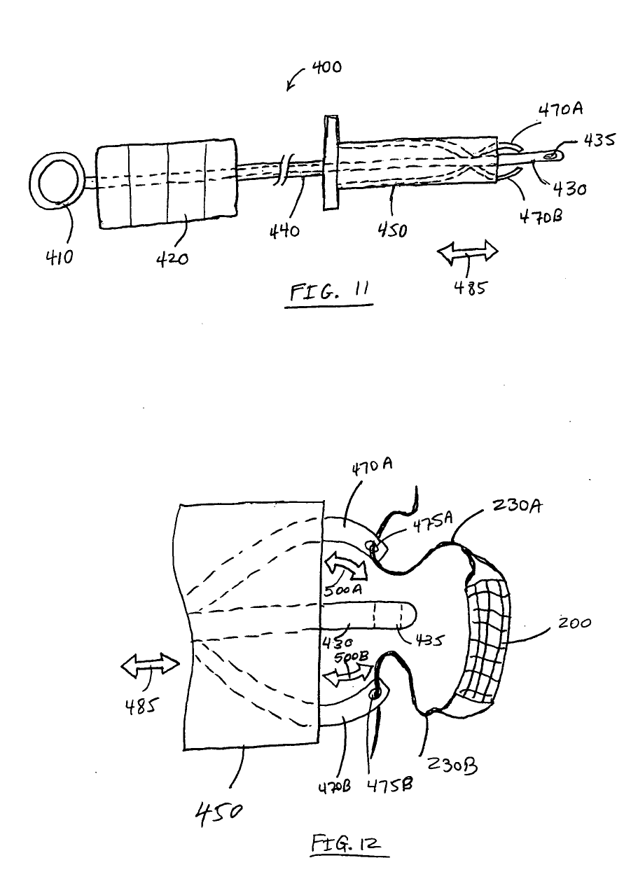

FIG. 11 illustrates an instrument 400 for threading the tails of the mesh implant into the respective perforations or openings in the spine's vertebral bodies. Instrument 400 includes handle 420, body 450, hollow shaft or tube 440, plate or guide or inserter 430, handle 410 (for plate 430), and a pair of needles or guides 470A and 470B.

Handle 420 provides a mechanism for the surgeon to hold and manipulate instrument 400. Handle 420 couples to shaft 440. Shaft 440 in turn couples to body 450. Thus, handle 420, shaft 440, and body 450 provide a channel through which plate 410 can slide back and forth.

Handle 410 couples to plate 430. Plate 430 can slide through handle 420 of the instrument, through shaft 440, and through body 450. Plate 430 has an opening 435. Tail 230A or tail 230B of the mesh implant can pass through opening 435.

Handle 410 provides a way for the surgeon to manipulate plate 430. By pushing in or pulling out handle 410, the surgeon can slide plate 430 through body 450. Pushing in handle 410 causes the end of plate 430 to protrude from body 450. Pulling out handle 410 causes the end of plate 430 to retract into body 450.

Needles 470A and 470B provide a mechanism for threading tails 230A and 230B (not shown in FIG. 11) through perforations 210A and 210B (not shown in FIG. 11) of vertebral bodies 100A and 100B (not shown in FIG. 11), respectively. Each of needles 470A and 470B has an opening (see FIG. 12) that allows a respective one of tails 230A and 230B to pass through it.

FIG. 12 depicts details of the operation of the instrument shown in FIG. 11. Plate 430 can slide in or out of body 450 along the direction indicated by arrow 485. Similarly, needles 470A and 470B can move along the directions indicated by arrows 500A and 500B, respectively. In one embodiment, needles 470A and 470B are made from nickel titanium to facilitate actuation along a curved path.

Note that FIG. 12 shows needles 470A and 470B each having an opening (labeled 475A and 475B, respectively). To use instrument 400, the surgeon threads tail 230A through opening 475A of needle 470A. Likewise, the surgeon threads tail 230B through opening 475B of needle 470B.

The surgeon also retracts plate 430 into body 450. The surgeon then inserts needle 470A (along with tail 230A) into perforation 210A (not shown explicitly) of vertebral body 100A (not shown explicitly) by pushing in body 450 in a posterior-to-anterior direction. Similarly, the surgeon inserts needle 470B (along with tail 230B) into perforation 210B (not shown explicitly) of vertebral body 100B (not shown explicitly).

Subsequently, the surgeon slides plate 430 in a posterior-to-anterior direction such that opening 435 of plate 430 becomes aligned or approximately aligned with openings 475A and 475B of needles 470A and 470B, respectively. By pushing needles 470A and 470B through, respectively, perforations 210A and 210B (not shown explicitly), the surgeon causes the threading of tails 230A and 230B through opening 435 of plate 430.

Once tails 230A and 230B thread through opening 435, the surgeon retracts needles 470A and 470B by pulling body 450 in an anterior-to-posterior direction. Needles 470A and 470B consequently retract from perforations 210A and 210B, leaving tails 230A and 230B threaded in opening 435 of plate 430.

The surgeon may then pull handle 410 (not shown in FIG. 12) in an anterior-to-posterior direction in order to retract plate 430 from the patient's body. As plate 430 retracts, it retrieves tails 230A and 230B of the mesh implant. The surgeon may then secure the mesh implant in its desired location, using a suitable technique, as described above in detail.

Various modifications and alternative embodiments of the invention in addition to those described here will be apparent to persons of ordinary skill in the art who have the benefit of the description of the invention. Accordingly, the manner of carrying out the invention as shown and described are to be construed as illustrative only.

Persons skilled in the art may make various changes in the shape, size, number, and/or arrangement of parts without departing from the scope of the invention described in this document. For example, persons skilled in the art may substitute equivalent elements for the elements illustrated and described here, or use certain features of the invention independently of the use of other features, without departing from the scope of the invention.

1. An implant, comprising a mesh, having a first tail and a second tail, the first and second tails configured to be threaded through respective first and second perforations in respective first and second vertebral bodies.

2. The implant according to claim 1, wherein the first perforation is made in an endplate of the first vertebral body, and wherein the second perforation is made in an endplate of the second vertebral body.

3. The implant according to claim 1, wherein the mesh comprises a natural biocompatible material.

4. The implant according to claim 1, wherein the mesh comprises a synthetic biocompatible material.

5. The implant according to claim 4, wherein the mesh comprises polyester.

6. The implant according to claim 1, wherein the first and second tails couple to the mesh via respective first and second loops.

7. The implant according to claim 1, wherein the first tail couples to a needle, and wherein the second tail couples to a needle.

8. The implant according to claim 1, wherein the mesh comprises permeable material.

9. The implant according to claim 1, wherein the mesh comprises impermeable material.

10. The implant according to claim 1, wherein the mesh comprises a therapeutic agent.

11. The implant according to claim 10, wherein the therapeutic agent comprises an anti-inflammatory agent.

12. The implant according to claim 10, wherein the therapeutic agent comprises an anti-adhesive agent.

13. The implant according to claim 10, wherein the therapeutic agent comprises a pro-adhesive agent.

14. The implant according to claim 10, wherein the therapeutic agent comprises at least one of: (a) an agent that inhibits pro-inflammatory cytokines; (b) an anti-enzymatic agent that inhibits degradation of tissue; (c) an agent that inhibits angiogenesis in a disc; and (d) a growth factor that promotes extracellular matrix production.

15. A method of repairing an annular defect in a spine by using a mesh implant, the mesh implant having a mesh coupled to first and second tails, the method comprising:
threading the first tail through a first perforation in a first vertebral body of the spine; and
threading the second tail through a second perforation in a second vertebral body of the spine.

16. The method according to claim 15, wherein the first and second tails are threaded by using first and second needles coupled, respectively, to the first and second tails.

17. The method according to claim 15, further comprising:
securing the first tail to the mesh implant; and
securing the second tail to the mesh implant.

18. The method according to claim 17, wherein the first and second tails are secured by tying first and second knots.

19. The method according to claim 17, wherein the first and second tails are secured by crimping.

20. The method according to claim 17, wherein the mesh is placed using a peri-annular placement technique.

21. The method according to claim 17, wherein the mesh is placed using an intra-annular placement technique.

22. The method according to claim 15, wherein threading the first tail further comprises using a trochar inserted into the first perforation in order to thread the first tail into the first perforation

23. The method according to claim 15, wherein threading the first tail further comprises using a first curved needle inserted into the first perforation in order to thread the first tail into the first perforation.

24. (canceled)

25. (canceled)

26. (canceled)

27. (canceled)

28. (canceled)

29. (canceled)

30. An apparatus, comprising a mesh implant for repairing an annular defect in a spine, the mesh implant comprising a therapeutic agent.

31. The apparatus according to claim 30, wherein the therapeutic agent comprises an anti-inflammatory agent.

32. The apparatus according to claim 30, wherein the therapeutic agent comprises an anti-adhesive agent.

33. The apparatus according to claim 30, wherein the therapeutic agent comprises a pro-adhesive agent.

34. The apparatus according to claim 30, wherein the therapeutic agent comprises at least one of: (a) an agent that inhibits pro-inflammatory cytokines; (b) an anti-enzymatic agent that inhibits degradation of tissue; (c) an agent that inhibits angiogenesis in a disc; and (d) a growth factor that promotes extracellular matrix production.
